(19) United States
(12) Patent Application Publication
(10) Pub. No.: US 2024/0382271 A1
(43) Pub. Date: Nov. 21, 2024

(54) SYSTEMS AND METHODS FOR CORRELATING ONE OR MORE MOTIONS OF AN ANATOMICAL ELEMENT

(71) Applicant: Medtronic Navigation, Inc., Lafayette,, CO (US)

(72) Inventor: Elliot C. Schmidt, Minneapolis, MN (US)

(21) Appl. No.: 18/631,107

(22) Filed: Apr. 10, 2024

Related U.S. Application Data
(60) Provisional application No. 63/466,571, filed on May 15, 2023.

(51) Int. Cl. A61B 34/00; A61B 34/10; A61B 34/20; A61B 34/30; A61B 90/00; A61M 25/01

(52) U.S. Cl. CPC A61B 34/25; A61B 34/30; A61B 2034/105; A61B 2034/2051; A61B 2034/2065; A61B 2090/367; A61M 2025/0166

(57) ABSTRACT

Systems and methods for correlating one or more motions of an anatomical element are provided. A plurality of first pose information of a first tracker corresponding to pose information of the first tracker can be received over a period of time. One or more first motion phases of a first motion of an anatomical element can be determined based on the plurality of first pose information. A plurality of second pose information of a second tracker corresponding to pose information of the second tracker can be received over the period of time. One or more second motion phases of a second motion of the anatomical element can be determined based on the plurality of second pose information.

### CROSS-REFERENCE TO RELATED APPLICATION

This application claims the benefit of and priority to U.S. Provisional Patent Application No. 63/466,571 filed May 15, 2023, the entire disclosure of which is incorporated by reference herein.

### BACKGROUND

The present disclosure is generally directed to tracking movement of an anatomical element, and relates more particularly to tracking movement of an anatomical element to correlate one or more movements or motions of the anatomical element.

Surgical robots may assist a surgeon or other medical provider in carrying out a surgical procedure, or may complete one or more surgical procedures autonomously. Imaging may be used by a medical provider for diagnostic and/or therapeutic purposes. Patient anatomy can change over time, particularly following placement of a medical implant in the patient anatomy.

### BRIEF SUMMARY

Example aspects of the present disclosure include:

A system for correlating one or more images with one or more phases of motion of an anatomical element according to at least one embodiment of the present disclosure comprises a processor; and a memory storing data for processing by the processor, the data, when processed, causing the processor to: receive a plurality of images over a period of time; receive a plurality of first pose information of a first tracker corresponding to pose information of the first tracker over the period of time; determine one or more first motion phases of a first motion of the anatomical element based on the plurality of first pose information; receive a plurality of second pose information of a second tracker corresponding to pose information of the second tracker over the period of time; determine one or more second motion phases of a second motion of the anatomical element based on the plurality of second pose information; and determine a subset of images from the plurality of images corresponding to a target first motion phase of the one or more first motion phases of the first motion and a target second motion phase of the one or more second motion phases of the second motion.

Any of the aspects herein, wherein the subset of images is a first subset of images, and wherein the memory stores further data for processing by the processor that, when processed, causes the processor to determine a second subset of images from the plurality of images corresponding to another target first motion phase of the one or more first motion phases of the first motion and another target second motion phase of the one or more second motion phases of the second motion.

Any of the aspects herein, wherein the memory stores further data for processing by the processor that, when processed, causes the processor to reconstruct a three-dimensional model based on at least one of the first subset of images or the second subset of images.

Any of the aspects herein, wherein the three-dimensional model is reconstructed in near real-time.

Any of the aspects herein, wherein the memory stores further data for processing by the processor that, when processed, causes the processor to display at least one of the first subset of images or the second subset of images.

Any of the aspects herein, wherein the first motion corresponds to a cardiac cycle and the second motion corresponds to a respiratory cycle.

Any of the aspects herein, further comprising a catheter positioned in the anatomical element, wherein the first tracker is positioned on the catheter and tracks movement of the catheter, and wherein the second tracker is positioned on the patient and tracks respiratory movement of the patient.

Any of the aspects herein, wherein the memory stores further data for processing by the processor that, when processed, causes the processor to obtain and display a subsequent plurality of images over a subsequent period of time at the target first motion phase and the target second motion phase.

A system for calibrating a tracker with one or more phases of motion of an anatomical element according to at least one embodiment of the present disclosure comprises a processor; and a memory storing data for processing by the processor, the data, when processed, causing the processor to: receive a plurality of first pose information of a first tracker over a period of time, the first tracker disposed in an anatomical element; determine one or more first motion phases of a first motion of the anatomical element based on the plurality of first pose information; receive a plurality of second pose information of a second tracker over the period of time, the second tracker tracking respiratory movement of a patient; determine one or more second motion phases of a second motion of the anatomical element based on the plurality of second pose information; and correlate the one or more first motion phases and the one or more second motion phases; and calibrate the second tracker based on the correlation of the one or more first motion phases and the one or more second motion phases.

Any of the aspects herein, wherein the memory stores further data for processing by the processor that, when processed, causes the processor to track the second tracker.

Any of the aspects herein, wherein the second tracker is external to the patient.

Any of the aspects herein, wherein the memory stores further data for processing by the processor that, when processed, causes the processor to: receive a second plurality of first pose information of the first tracker over a second period of time; determine a second one or more first motion phases of a first motion of the anatomical element based on the second plurality of first pose information; receive a second plurality of second pose information of the second tracker over the second period of time; determine a second one or more second motion phases of a second motion of the anatomical element based on the second plurality of second pose information; correlate the second one or more first motion phases and the second one or more second motion phases; and recalibrate the second tracker based on the correlation of the second one or more first motion phases and the second one or more second motion phases.

Any of the aspects herein, wherein the memory stores further data for processing by the processor that, when processed, causes the processor to: receive a plurality of images over the period of time; and determine a subset of images from the plurality of images corresponding to a target first motion phase of the one or more first motion phases of the first motion and a target second motion phase of the one or more second motion phases of the second motion.

Any of the aspects herein, wherein the memory stores further data for processing by the processor that, when processed, causes the processor to reconstruct a three-dimensional model based on at least one of the first subset of images or the second subset of images.

Any of the aspects herein, wherein the memory stores further data for processing by the processor that, when processed, causes the processor to obtain and display a subsequent plurality of images over a subsequent period of time at the target first motion phase and the target second motion phase.

Any of the aspects herein, wherein the first motion corresponds to a cardiac cycle.

Any of the aspects herein, wherein the plurality of second pose information is received as sensor data from at least one of an electromagnetic tracker or an impedance tracker.

Any of the aspects herein, wherein the plurality of second pose information is received from a navigation system configured to track the second tracker.

Any of the aspects herein, wherein the memory stores further data for processing by the processor that, when processed, causes the processor to determine another subset of images from the plurality of images corresponding to another target first motion phase of the one or more first motion phases of the first motion and another target second motion phase of the one or more second motion phases of the second motion.

Any of the aspects herein, wherein the memory stores further data for processing by the processor that, when processed, causes the processor to: receive a plurality of third pose information of a third tracker over the period of time, the third tracker positioned on the patient and spaced from the second tracker; and correlate the third tracker and the second tracker.

A system for correlating one or more images with one or more phases of motion of an anatomical element according to at least one embodiment of the present disclosure comprises a first tracker disposed on a catheter, the catheter positioned in the anatomical element; as second tracker disposed on a patient; a processor; and a memory storing data for processing by the processor, the data, when processed, causing the processor to: receive a plurality of first pose information of the first tracker over a period of time, the first pose information corresponding to pose information of the catheter; determine one or more first motion phases of a first motion of the anatomical element based on the plurality of first pose information; receive a plurality of second pose information of the second tracker over the period of time; determine one or more second motion phases of a second motion of the anatomical element based on the plurality of second pose information; and correlate the one or more first motion phases and the second motion phases.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages of the present disclosure will become apparent to those skilled in the art upon consideration of the embodiment descriptions provided hereinbelow.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a block diagram of a system according to at least one embodiment of the present disclosure;

FIG. 2A is a schematic of a patient respiratory cycle according to at least one embodiment of the present disclosure;

FIG. 2B are X-ray images of the patient respiratory cycle according to at least one embodiment of the present disclosure;

FIG. 3 is a flowchart according to at least one embodiment of the present disclosure; and

FIG. 4 is a flowchart according to at least one embodiment of the present disclosure.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia Geforce RTX 2000-series processors, Nvidia Geforce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

Anatomical models such as, for example, models of a heart, (created through, for example, image segmenting of an ultrasound, registration of a computer tomography (CT) scan or an magnetic resonance imaging (MRI) scan, electro-anatomical mapping, etc.) require registration with the patient's anatomy (e.g., a patient's heart) and navigational equipment used during a surgical procedure (e.g., electromagnetic (EM) sensors/emitters, impedance patches, imaging equipment, etc.). The position of the patient's anatomy and in particular, a patient's thoracic anatomy (e.g., heart) is often relatively dynamic during a procedure due at least in part to a patient's respiration. Thus, to improve accuracy of navigation systems and the registration of the anatomical models, it is desirable to account for the patient's respiration.

According to at least one embodiment of the present disclosure, an external reference or fiducial patch(s) is tracked using a technology such as EM or impedance. The patch(es) may be placed on a portion of the patient with significant respiratory motion (e.g. sternal, subxiphoid, etc.). The external reference may be coupled with an internal catheter/device in the anatomy of interest (e.g.) catheter inside the heart). The external reference would be used to identify respiratory state, speed, and magnitude (deep fast breath in with lungs full, slow, steady breathing out with lungs 50% empty, etc.).

While the external reference is monitoring the state of respiration, simultaneously the internal device/catheter will be monitoring positional changes while at rest (e.g., not being actively manipulated). This would allow for calibration between movement of the external reference and respirational induced positional change in the target anatomy. To enhance the calibration process, it may be useful to have the patient conduct some simple breathing exercises. For example, the patient may be instructed to breath in deeply and suddenly and the movement of the external reference and internal device may be monitored during such breathing. Then, for example, the patient may be instructed to exhale slowly and completely while again tracking the position of the external reference and internal device.

The systems and methods described in the present disclosure beneficially improve the accuracy of navigation systems in the cardiac space while reducing costs and remaining non-invasive.

Embodiments of the present disclosure provide technical solutions to one or more of the problems of (1) determining motion of an anatomical element due to two or more cyclic motions (e.g., respiratory cycle and cardiac cycle), (2) improving navigation during a surgical procedure, and (3) increasing patient safety.

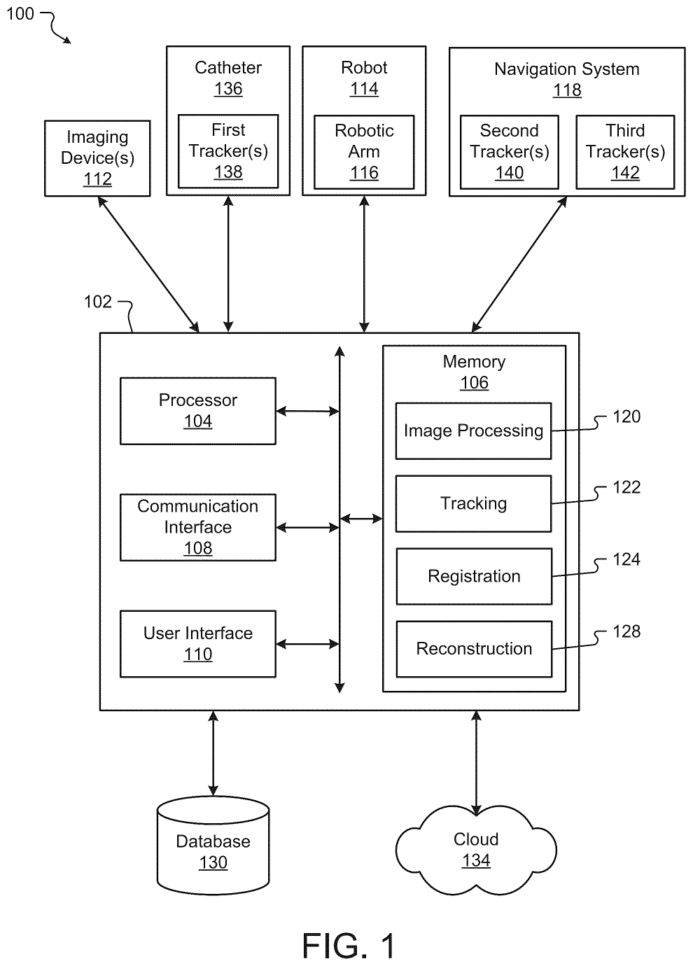

Turning first to FIG. 1, a block diagram of a system 100 according to at least one embodiment of the present disclosure is shown. The system 100 may be used to track movement of an anatomical element, determine one or more phases of motions of the anatomical element, correlate the one or more phases of different motions, and/or carry out one or more other aspects of one or more of the methods disclosed herein. The system 100 comprises a computing device 102, one or more imaging devices 112, a catheter 136 (trackable with a first tracker 138), a robot 114, a navigation system 118, a database 130, and/or a cloud or other network 134. Systems according to other embodiments of the present disclosure may comprise more or fewer components than the system 100. For example, the system 100 may not include the imaging device 112, the robot 114, the navigation system 118, one or more components of the computing device 102, the database 130, and/or the cloud 134.

The computing device 102 comprises a processor 104, a memory 106, a communication interface 108, and a user interface 110. Computing devices according to other embodiments of the present disclosure may comprise more or fewer components than the computing device 102.

The processor 104 of the computing device 102 may be any processor described herein or any similar processor. The processor 104 may be configured to execute instructions stored in the memory 106, which instructions may cause the processor 104 to carry out one or more computing steps utilizing or based on data received from the imaging device 112, the first tracker 138, the second tracker 140, the robot 114, the navigation system 118, the database 130, and/or the cloud 134.

The memory 106 may be or comprise RAM, DRAM, SDRAM, other solid-state memory, any memory described herein, or any other tangible, non-transitory memory for storing computer-readable data and/or instructions. The memory 106 may store information or data useful for completing, for example, any step of the methods 300 and/or 400 described herein, or of any other methods. The memory 106 may store, for example, instructions and/or machine learning models that support one or more functions of the robot 114. For instance, the memory 106 may store content (e.g., instructions and/or machine learning models) that, when executed by the processor 104, enable image processing 120, tracking 122, registration 124, and/or reconstruction 128.

The image processing 120 enables the processor 104 to process image data of an image (received from, for example, the imaging device 112, an imaging device of the navigation system 118, or any imaging device) for the purpose of, for example, identifying information about anatomical elements and/or objects depicted in the image. The information may comprise, for example, identification of hard tissue and/or soft tissues, a boundary between hard tissue and soft tissue, a boundary of hard tissue and/or soft tissue, identification of a surgical tool, etc. The image processing 120 may, for example, identify hard tissue, soft tissue, and/or a boundary of the hard tissue and/or soft tissue by determining a difference in or contrast between colors or grayscales of image pixels. For example, a boundary between the hard tissue and the soft tissue may be identified as a contrast between lighter pixels and darker pixels.

The tracking 122 enables the processor 104 to track the first tracker 138 and/or the second tracker 140 which may comprise, for example, EM and/or inertial measurement unit (IMU) trackers. The tracking 122 may, for example, enable the processor 104 to receive sensor data from the first tracker 138 and determine pose information (e.g., position and orientation) of the catheter 136 from the sensor data. The tracking 122 may also, for example, enable the processor 104 to track the second tracker 140 after the second tracker 140 has been calibrated to one or more motions of an anatomical element such as, for example, a heart.

The registration 124 enables the processor 104 to correlate an image with another image. The registration 124 may enable the processor 104 to also correlate identified anatomical elements and/or objects in one image with identified anatomical elements and/or objects in another image. The registration 124 may also enable information about the anatomical elements and the objects (e.g., the catheter 136, for example) to be obtained and measured.

The reconstruction 128 enables the processor 104 to generate a three-dimensional (3D) representation of one or more anatomical elements and/or one or more objects based on one or more image(s) received by the processor 104. Generating the 3D representation by the processor 104 may include determining a surface representation or virtual boundary of the one or more anatomical elements and/or the one or more objects depicted in image(s) received by the processor 104 and based on corresponding image pose information. More specifically, in some embodiments, each image may be positioned adjacent to another image based on the respective corresponding image pose information and a surface representation may be formed based the relative position of surfaces depicted in each image. In some embodiments, the surface representation may be a virtual mesh. The virtual mesh may comprise, for example, a set of polygonal faces that, when taken together, form a surface covering of a virtual object. The set of polygonal faces may be connected at their edges and vertices to define a shape of the virtual object.

Such content, if provided as instructions, may, in some embodiments, be organized into one or more applications, modules, packages, layers, or engines. Alternatively or additionally, the memory 106 may store other types of content or data (e.g., machine learning models, artificial neural networks, deep neural networks, etc.) that can be processed by the processor 104 to carry out the various method and features described herein. Thus, although various contents of memory 106 may be described as instructions, it should be appreciated that functionality described herein can be achieved through use of instructions, algorithms, and/or machine learning models. The data, algorithms, and/or instructions may cause the processor 104 to manipulate data stored in the memory 106 and/or received from or via the imaging device 112, the first tracker 138, the second tracker 140, the robot 114, the database 130, and/or the cloud 134.

The computing device 102 may also comprise a communication interface 108. The communication interface 108 may be used for receiving image data or other information from an external source (such as the imaging device 112, the first tracker 138, the second tracker 140, the robot 114, the navigation system 118, the database 130, the cloud 134, and/or any other system or component not part of the system 100), and/or for transmitting instructions, images, or other information to an external system or device (e.g., another computing device 102, the imaging device 112, the first tracker 138, the second tracker 140, the robot 114, the navigation system 118, the database 130, the cloud 134, and/or any other system or component not part of the system 100). The communication interface 108 may comprise one or more wired interfaces (e.g., a USB port, an Ethernet port, a Firewire port) and/or one or more wireless transceivers or interfaces (configured, for example, to transmit and/or receive information via one or more wireless communication protocols such as 802.11a/b/g/n, Bluetooth, NFC, ZigBee, and so forth). In some embodiments, the communication interface 108 may be useful for enabling the device 102 to communicate with one or more other processors 104 or computing devices 102, whether to reduce the time needed to accomplish a computing-intensive task or for any other reason.

The computing device 102 may also comprise one or more user interfaces 110. The user interface 110 may be or comprise a keyboard, mouse, trackball, monitor, television, screen, touchscreen, and/or any other device for receiving information from a user and/or for providing information to a user. The user interface 110 may be used, for example, to receive a user selection or other user input regarding any step of any method described herein. Notwithstanding the foregoing, any required input for any step of any method described herein may be generated automatically by the system 100 (e.g., by the processor 104 or another component of the system 100) or received by the system 100 from a source external to the system 100. In some embodiments, the user interface 110 may be useful to allow a surgeon or other user to modify instructions to be executed by the processor 104 according to one or more embodiments of the present disclosure, and/or to modify or adjust a setting of other information displayed on the user interface 110 or corresponding thereto.

Although the user interface 110 is shown as part of the computing device 102, in some embodiments, the computing device 102 may utilize a user interface 110 that is housed separately from one or more remaining components of the computing device 102. In some embodiments, the user interface 110 may be located proximate one or more other components of the computing device 102, while in other embodiments, the user interface 110 may be located remotely from one or more other components of the computer device 102.

The imaging device 112 may be operable to image anatomical feature(s) (e.g., a bone, veins, tissue, etc.) and/or other aspects of patient anatomy to yield image data (e.g., image data depicting or corresponding to a bone, veins, tissue, etc.). “Image data” as used herein refers to the data generated or captured by an imaging device 112, including in a machine-readable form, a graphical/visual form, and in any other form. In various examples, the image data may comprise data corresponding to an anatomical feature of a patient, or to a portion thereof. The image data may be or comprise a preoperative image, an intraoperative image, a postoperative image, or an image taken independently of any surgical procedure. In some embodiments, a first imaging device 112 may be used to obtain first image data (e.g., a first image) at a first time, and a second imaging device 112 may be used to obtain second image data (e.g., a second image) at a second time after the first time. The imaging device 112 may be capable of taking a 2D image or a 3D image to yield the image data. The imaging device 112 may be or comprise, for example, an ultrasound scanner (which may comprise, for example, a physically separate transducer and receiver, or a single ultrasound transceiver), an O-arm, a C-arm, a G-arm, or any other device utilizing X-ray-based imaging (e.g., a fluoroscope, a CT scanner, or other X-ray machine), a magnetic resonance imaging (MRI) scanner, an optical coherence tomography (OCT) scanner, an endoscope, a microscope, an optical camera, a thermographic camera (e.g., an infrared camera), a radar system (which may comprise, for example, a transmitter, a receiver, a processor, and one or more antennae), or any other imaging device 112 suitable for obtaining images of an anatomical feature of a patient. The imaging device 112 may be contained entirely within a single housing, or may comprise a transmitter/emitter and a receiver/detector that are in separate housings or are otherwise physically separated.

In some embodiments, the imaging device 112 may comprise more than one imaging device 112. For example, a first imaging device may provide first image data and/or a first image, and a second imaging device may provide second image data and/or a second image. In still other embodiments, the same imaging device may be used to provide both the first image data and the second image data, and/or any other image data described herein. The imaging device 112 may be operable to generate a stream of image data. For example, the imaging device 112 may be configured to operate with an open shutter, or with a shutter that continuously alternates between open and shut so as to capture successive images. For purposes of the present disclosure, unless specified otherwise, image data may be considered to be continuous and/or provided as an image data stream if the image data represents two or more frames per second.

The catheter 136 may be any catheter 136 that can be inserted and positioned in a patient and more specifically, may be inserted into an anatomical element of the patient. For example, the catheter 136 may be inserted into a patient's heart, though it will be appreciated that the catheter 136 can be positioned in any anatomical element or region of the patient (e.g., intrathecally in the spinal region of the patient, in the brain region of the patient, etc.). In some instances, the catheter 136 can be implanted in the patient. In some embodiments, the catheter 136 may enable fluid sampling from the patient and/or delivery of therapeutics to the patient.

The first tracker 138 may comprise one or more trackers. The first tracker 138 may include one or more sensors disposed on the catheter 136 and configured to track a pose and/or movement of the catheter 136. The first tracker 138 may comprise, for example, EM and/or IMU sensors. In other embodiments, the first tracker 138 may include one or more or any combination of components that are electrical, mechanical, electro-mechanical, magnetic, electromagnetic, or the like. For example, the first tracker 138 may alternatively or additionally include infrared tracking, light diffraction tracking (e.g., optic fiber), and/or impedance tracking. The first tracker 138 may be positioned at a tip of the catheter 136, though in other embodiments, the first tracker 138 may be positioned on any portion of the catheter 136. The first tracker 138 may include a plurality of sensors and each sensor may be positioned at the same location or a different location as any other sensor. In some embodiments, the first tracker 138 may include a memory for storing sensor data. In still other examples, the first tracker 138 may output signals (e.g., sensor data) to one or more sources (e.g., the computing device 102, the navigation system 114, and/or the robot 116). For example, the first tracker 138 may send the data to the computing device 102 when the first tracker 138 detects movement of the catheter 136. Further, in some embodiments, the first tracker 138 may send data to the computing device 102 to display on the user interface 110 or otherwise notify the surgeon or operator of movement of the catheter 136.

Similar to the first tracker 128, the second tracker 140 and/or the third tracker 142 may comprise one or more sensors. It will be appreciated that alternatively or additionally, the second tracker 140 may comprise one or more reference markers. The second tracker 140 and/or the third tracker 142 may include one or more trackers. The second tracker 140 and/or the third tracker 142 may be disposed on a patient (whether external or internal to the patient) and may be configured to track movement of the anatomical element relative to, for example, respiratory movement. The second tracker 140 and/or the third tracker 142 may comprise, for example, EM and/or IMU sensors. In other embodiments, the second tracker 140 and/or the third tracker 142 may include one or more or any combination of components that are electrical, mechanical, electro-mechanical, magnetic, electromagnetic, or the like. For example, the second tracker 140 and/or the third tracker 142 may additionally or alternatively include infrared tracking, light diffraction tracking (e.g., optic fiber), and/or impedance tracking. The second tracker 140 and/or the third tracker 142 may include a plurality of sensors and each sensor may be positioned at the same location or a different location as any other sensor. In some embodiments, the second tracker 140 and/or the third tracker 142 may include a memory for storing sensor data. In still other examples, the second tracker 140 and/or the third tracker 142 may output signals (e.g., sensor data) to one or more sources (e.g., the computing device 102, the navigation system 114, and/or the robot 116). For example, the second tracker 140 and/or the third tracker 142 may send the data to the computing device 102 when the second tracker 140 and/or the third tracker 142 detects movement of the anatomical element. Further, in some embodiments, the second tracker 140 and/or the third tracker 142 may send data to the computing device 102 to display on the user interface 110 or otherwise notify the surgeon or operator of movement of the second tracker 140 and/or the third tracker 142.

The robot 114 may be any surgical robot or surgical robotic system. The robot 114 may be or comprise, for example, the Mazor X™ Stealth Edition robotic guidance system. The robot 114 may be configured to position the imaging device 112 at one or more precise position(s) and orientation(s), and/or to return the imaging device 112 to the same position(s) and orientation(s) at a later point in time. The robot 114 may additionally or alternatively be configured to manipulate a surgical tool (whether based on guidance from the navigation system 118 or not) to accomplish or to assist with a surgical task. In some embodiments, the robot 114 may be configured to hold and/or manipulate an anatomical element during or in connection with a surgical procedure. The robot 114 may comprise one or more robotic arms 116. In some embodiments, the robotic arm 116 may comprise a first robotic arm and a second robotic arm, though the robot 114 may comprise more than two robotic arms. In some embodiments, one or more of the robotic arms 116 may be used to hold and/or maneuver the imaging device 112. In embodiments where the imaging device 112 comprises two or more physically separate components (e.g., a transmitter and receiver), one robotic arm 116 may hold one such component, and another robotic arm 116 may hold another such component. Each robotic arm 116 may be positionable independently of the other robotic arm. The robotic arms 116 may be controlled in a single, shared coordinate space, or in separate coordinate spaces. The robotic arm(s) 116 may comprise one or more sensors that enable the processor 104 (or a processor of the robot 114) to determine a precise pose in space of the robotic arm (as well as any object or element held by or secured to the robotic arm).

In some embodiments, reference markers (e.g., navigation markers) may be placed on the robot 114 (including, e.g., on the robotic arm 116), the imaging device 112, the patient, or any other object in the surgical space. The reference markers may be tracked by the navigation system 118, and the results of the tracking may be used by the robot 114 and/or by an operator of the system 100 or any component thereof. In some embodiments, the navigation system 118 can be used to track other components of the system (e.g., imaging device 112) and the system can operate without the use of the robot 114 (e.g., with the surgeon manually manipulating the imaging device 112 and/or one or more surgical tools, based on information and/or instructions generated by the navigation system 118, for example).

The navigation system 118 may provide navigation for a surgeon and/or a surgical robot during an operation. The navigation system 118 may be any now-known or future-developed navigation system, including, for example, the Medtronic StealthStation™ S8 surgical navigation system or any successor thereof. The navigation system 118 may include one or more cameras or other sensor(s) for tracking the first tracker 138, the second tracker 140, one or more reference markers, navigated trackers, or other objects within the operating room or other room in which some or all of the system 100 is located. The one or more cameras may be optical cameras, infrared cameras, or other cameras. In some embodiments, the navigation system 118 may comprise one or more electromagnetic sensors or trackers or inertial measurement unit trackers (e.g., the first tracker 138 and/or the second tracker 140). In various embodiments, the navigation system 118 may be used to track a position and orientation (e.g., a pose) of the catheter 136 via the first tracker 138, the second tracker 140, the imaging device 112, the robot 114 and/or robotic arm 116, and/or one or more surgical tools (or, more particularly, to track a pose of a navigated tracker attached, directly or indirectly, in fixed relation to the one or more of the foregoing). The navigation system 118 may include a display for displaying one or more images from an external source (e.g., the computing device 102, imaging device 112, or other source) or for displaying an image and/or video stream from the one or more cameras or other sensors of the navigation system 118. The navigation system 118 may be configured to provide guidance to a surgeon or other user of the system 100 or a component thereof, to the robot 114, or to any other element of the system 100 regarding, for example, a pose of one or more anatomical elements, whether or not a tool is in the proper trajectory, and/or how to move a tool into the proper trajectory to carry out a surgical task according to a preoperative or other surgical plan.

The database 130 may store information that correlates one coordinate system to another (e.g., one or more robotic coordinate systems to a patient coordinate system and/or to a navigation coordinate system). The database 130 may additionally or alternatively store, for example, one or more surgical plans (including, for example, pose information about a target and/or image information about a patient's anatomy at and/or proximate the surgical site, for use by the robot 114, the navigation system 118, and/or a user of the computing device 102 or of the system 100); one or more images useful in connection with a surgery to be completed by or with the assistance of one or more other components of the system 100; and/or any other useful information. The database 130 may be configured to provide any such information to the computing device 102 or to any other device of the system 100 or external to the system 100, whether directly or via the cloud 134. In some embodiments, the database 130 may be or comprise part of a hospital image storage system, such as a picture archiving and communication system (PACS), a health information system (HIS), and/or another system for collecting, storing, managing, and/or transmitting electronic medical records including image data.

The cloud 134 may be or represent the Internet or any other wide area network. The computing device 102 may be connected to the cloud 134 via the communication interface 108, using a wired connection, a wireless connection, or both. In some embodiments, the computing device 102 may communicate with the database 130 and/or an external device (e.g., a computing device) via the cloud 134.

The system 100 or similar systems may be used, for example, to carry out one or more aspects of any of the methods 300 and/or 400 described herein. The system 100 or similar systems may also be used for other purposes.

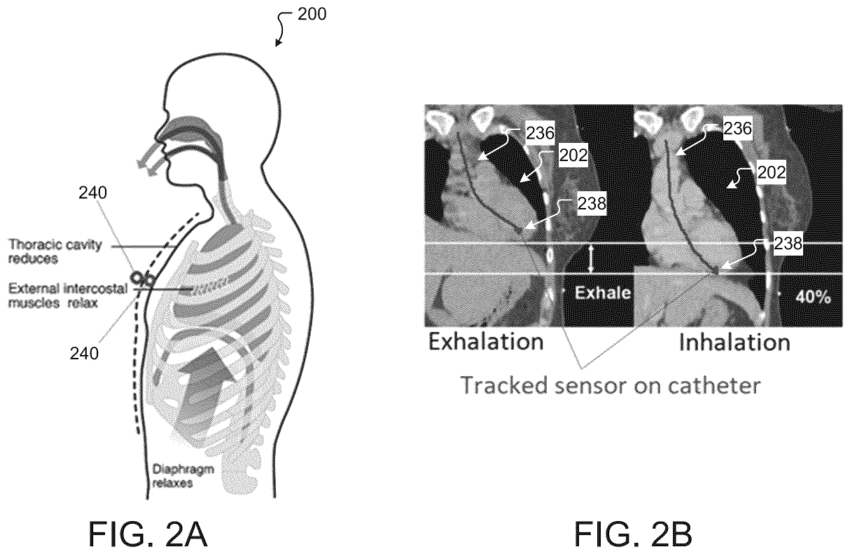

Turning to FIGS. 2A and 2B, a schematic of a patient's 200 respiratory cycle and X-ray images of the patient's 200 respiratory cycle are respectively shown. As shown in FIG. 2A, the second tracker 140 is shown in a first pose and a second pose. The first pose may correspond to a pose of the second tracker 140 during inspiration of a respiratory cycle and the second pose may correspond to a pose of the second tracker 140 during expiration pf the respiratory cycle. As also shown, during inspiration, the thoracic cavity reduces and during expiration, the external intercostal muscles relaxes. Thus, the second tracker 140 may be used to track the respiratory cycle of the patient 200, which may then be correlated with a cardiac cycle of the patient 200.

As shown in FIG. 2B, the first tracker 138 is shown on the catheter 136 in a third pose and a fourth pose and disposed within an anatomical element 202. The third pose may correspond to a pose of the first tracker 138 during expiration and the fourth pose may correspond to a pose of the first tracker 138 during inspiration. As shown, the first tracker 138 may move in one or more directions between inspiration and expiration. Such information may be correlated with movement of the second tracker 140 as shown in FIG. 2A so that the second tracker 140 can be used to track the anatomical element 202. More specifically, the first pose may be correlated with the fourth pose and the second pose may be correlated with the third pose. Such correlation may be used to calibrate the second tracker 140 such that the second tracker 140 can be tracked and used to track movement of the anatomical element 202 without tracking the first tracker 138. In other words, the second tracker 140 can be used to track movement of the anatomical element 202 through both the cardiac cycle and the respiratory cycle.

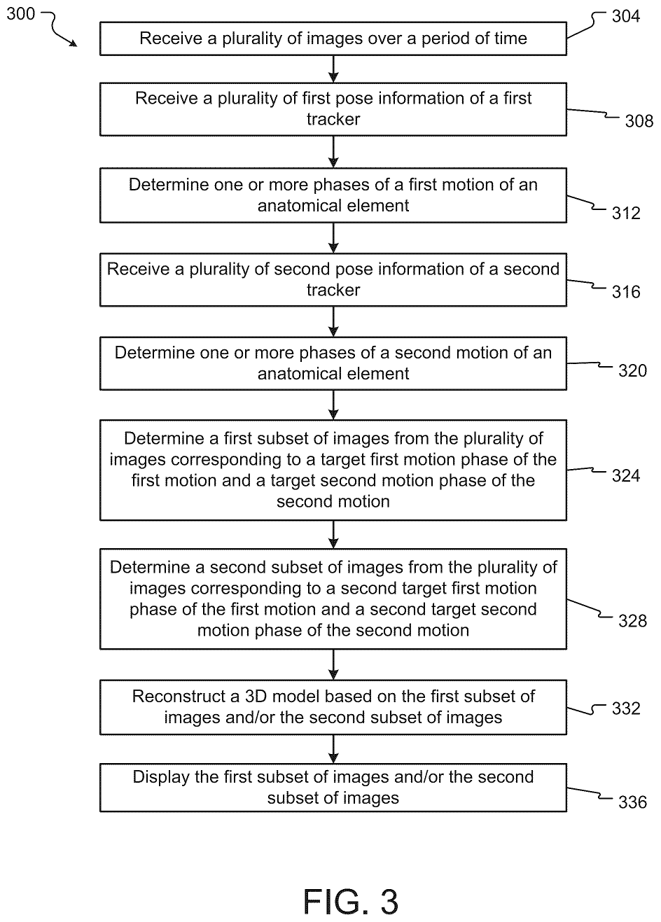

FIG. 3 depicts a method 300 that may be used, for example, for determining one or more phases of motions of an anatomical element (e.g., a heart).

The method 300 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 300. The at least one processor may perform the method 300 by executing elements stored in a memory such as the memory 106. The elements stored in memory and executed by the processor may cause the processor to execute one or more steps of a function as shown in method 300. One or more portions of a method 300 may be performed by the processor executing any of the contents of memory, such as image processing 120, tracking 122, registration 124, and/or reconstruction 128.

The method 300 comprises receiving a plurality of images over a period of time (step 304). The plurality of images may be received or obtained from an imaging device such as the imaging device 112 and may be stored in the memory of a computing device such as the computing device 102. An imaging device may be any imaging device such as an MRI scanner, a CT scanner, any other X-ray based imaging device, or an ultrasound imaging device. The image may also be generated by and/or uploaded to any other component of a system such as the system 100. In some embodiments, the image may be indirectly received via any other component of the system or a node of a network to which the system is connected.

The image may be a 2D image or a 3D image or a set of 2D and/or 3D images. The image may depict a patient's anatomy or portion thereof and may include at least a portion of the anatomical element and/or the catheter. It will be appreciated that in some embodiments, the image may also depict a reference marker (which may be in some embodiments a second tracker such as the second tracker 140). In some embodiments, the image may depict multiple anatomical elements associated with the patient anatomy, including incidental anatomical elements (e.g., ribs or other anatomical objects on which a surgery or surgical procedure will not be performed) in addition to target anatomical elements (e.g., vertebrae or other anatomical objects on which a surgery or surgical procedure is to be performed). The image may comprise various features corresponding to the patient's anatomy and/or anatomical elements (and/or portions thereof), including gradients corresponding to boundaries and/or contours of the various depicted anatomical elements, varying levels of intensity corresponding to varying surface textures of the various depicted anatomical elements, combinations thereof, and/or the like. The image may depict any portion or part of patient anatomy and may include, but is in no way limited to, one or more vertebrae, ribs, lungs, soft tissues (e.g., skin, tendons, muscle fiber, etc.), a patella, a clavicle, a scapula, combinations thereof, and/or the like.

Each image may be processed by the processor using an image processing such as the image processing 120 to identify anatomical elements and/or the reference marker in the image. In some embodiments, feature recognition may be used to identify a feature of the anatomical element or the reference marker. For example, a contour of a vertebrae, femur, or other bone may be identified in the image. In other embodiments, the image processing algorithm may use artificial intelligence or machine learning to identify the anatomical element and/or the reference marker. In such embodiments, a plurality of training images may be provided to the processor, and each training image may be annotated to include identifying information about a reference marker and/or an anatomical element in the image.

The method 300 also comprises receiving a plurality of first pose information of a first tracker (step 308). The plurality of first pose information is received from the first tracker which may be the same as or similar to the first tracker 138. The first tracker may be disposed on, for example, a catheter such as the catheter 136 and the plurality of first pose information may correspond to a plurality of poses of the catheter. The catheter may be disposed in an anatomical element such as the anatomical element 202. The anatomical element may comprise, for example, a patient's heart, though it will be appreciated that in other embodiments the anatomical element may comprise any anatomical element such as, for example, a patient's lung(s).

The plurality of first pose information may be received as sensor data and may be stored in, for example, memory such as the memory 106 and/or a database such as the database 130. The sensor data may be received wirelessly from the first tracker by, for example, a navigation system such as the navigation system 118. The sensor data can be received continuously in real-time or near real-time. The sensor data can also be received at a predetermined interval or based on user input. The sensor data may be used by, for example, the processor (or a processor of the navigation system) to obtain the plurality of pose information. The pose information may include information such as a position and orientation of the first tracker (and thus, the catheter), coordinates of the first tracker, etc.

The method 300 also comprises determining one or more phases of a first motion of an anatomical element (step 312). The one or more phases of motion may be determined by, for example, the processor mapping or graphing the movement of the first tracker (e.g., the catheter) and identifying repeated patterns of the movement. The processor may then correlate the patterns of the movement with the one or more phases of motion. For example, in embodiments where the anatomical element comprises the heart or an organ adjacent to or near the heart, the repeated patterns may correspond to one or more phases of the heartbeat or cardiac cycle. More specifically, the one or more phases may comprise at least a diastole motion and a systole motion, which can be identified and correlated to the patterns of movement. The processor may also use artificial intelligence or machine learning to identify the one or more phases. In such embodiments, a plurality of training poses may be provided to the processor, and each set of training poses may be annotated to include identifying information about the corresponding one or more phases.

It is beneficial to determine the one or more phases (e.g., the diastole motion and/or the systole motion) as images can be taken during a target phase (or phases) that produce higher quality images than images taken during an undesired phase, as will be described in detail below.

The method 300 also comprises receiving a plurality of second pose information of a second tracker (step 316). The plurality of second pose information is received from the second tracker which may be the same as or similar to the second tracker 140. The second tracker may be disposed on, for example, a patient such as the patient 200. The second tracker may be disposed on a surface of the patient (e.g., external to the patient) or implanted in the patient. The plurality of second pose information may be received as sensor data and may be stored in, for example, the memory and/or the database. The sensor data may be received wirelessly from the second tracker by, for example, the navigation system. The sensor data can be received continuously in real-time or near real-time. The sensor data can also be received at a predetermined interval or based on user input. The sensor data may be used by, for example, the processor to obtain the plurality of pose information. The pose information may include information such as a position and orientation of the second tracker, coordinates of the second tracker, etc.

The method 300 also comprises determining one or more phases of a second motion of the anatomical element (step 320). The step 420 is the same as or similar to the step 312 of the method 300 described above, except that the second motion may correspond to a breathing or respiratory cycle. Further, the one or more phases may correspond to inspiration or expiration (e.g., inhalation or exhalation, respectively) during a respiratory cycle. In such embodiments, the second motion (e.g., respiratory motion) may be determined from the plurality of second pose information.

The method 300 also comprises determining a first subset of images from the plurality of images corresponding to a target first motion phase of the first motion and a target second motion phase of the second motion (step 324). The first subset of images may be determined by, for example, the processor determining each image that corresponds to the first phase of the first motion (e.g., a first phase of a cardiac cycle) and the first phase of the second motion (e.g., a first phase of a respiratory cycle). More specifically, the processor may correlate the plurality of images taken over the period of time with the plurality of first pose information and the plurality of second pose information over the period of time. Thus, each image can be correlated to a phase of the first motion (e.g., cardiac motion) and a phase of the second motion (e.g., respiratory motion) based on the corresponding plurality of first pose information and plurality of second pose information. Images of the same target phase of the first motion and the second motion may then be grouped together.

The method 300 also comprises determining a second subset of images from the plurality of images corresponding to a second target first motion phase of the first motion and a second target second motion phase of the second motion (step 328). The step 428 may be the same as or similar to the step 424, except that the second subset of images is different from the first subset of images.

The method 300 also comprises reconstructing a 3D model (step 332). The 3D model may be reconstructed or updated based on the plurality of images received in, for example, the step 304. In some embodiments, the plurality of images may be registered to the 3D model by the processor using a registration such as the registration 124. After registration, the 3D model may be reconstruction by the processor using a reconstruction such as the reconstruction 128. As previously described, generating the 3D representation by the processor 104 may include determining a surface representation or virtual boundary of the one or more anatomical elements and/or the one or more objects depicted in image(s) and image pose information (e.g., pose information of the imaging device when the plurality of images are taken). The image pose information may be obtained from, for example, a robot such as the robot 114 supporting the imaging device. In some embodiments, each image may be positioned adjacent to another image based on the respective corresponding image pose information and a surface representation may be formed based the relative position of surfaces depicted in each image. In some embodiments, the surface representation may be a virtual mesh. The virtual mesh may comprise, for example, a set of polygonal faces that, when taken together, form a surface covering of a virtual object. The set of polygonal faces may be connected at their edges and vertices to define a shape of the virtual object.

The 3D model may be reconstructed using images from a target phase. For example, a first image from a first angle and a second image from a second angle may be taken at the same target phase such that portions of the anatomical element reconstructed from the first image and the second image are of similar position, size, and shape. In other words, if the first image and the second image are taken at different phases, the portions of the anatomical element depicted in the first image and the second image may be of different positions, sizes, and/or shapes and may not match up to each other. Thus, it is beneficial to obtain images from the same target phase(s) to reconstruct the 3D model. It will be appreciated that images from more than one phase can be used to reconstruct the 3D model in multiple phases.

The method 300 also comprises displaying the first subset of images and/or the second subset of images (step 336). The first subset of images and/or the second subset of images may be displayed on a user interface such as the user interface 110. The image(s) may be displayed at target phase(s) of motion(s) such as, for example, by strobing or freezing images (which may be, for example, ultrasound images) at the target phase(s) and/or matching the target phase(s) of a 3D image (e.g., CT or MRI) at target phase(s). Such selective displaying of the image may visually aid a user such as a surgeon or other medical provider during a surgical procedure such as, for example, implanting an implantable device. More specifically, by displaying the image at a target first phase of a first motion and a target first phase of a second motion, the image of the anatomical element may appear to be near stationary to a user such as, for example, a surgeon or other medical provider during a surgical procedure. Without the selective display at the target phase(s), the anatomical element would appear to be constantly moving due to the cyclic motion and the respiratory motion, which may result in difficulty in visualizing the anatomical element during the surgical procedure.

It will be appreciated that any step and any combination of steps of the method 300 may be repeated. For example, the step 324 or 328 may be repeated for a third subset of images or any subsequent set(s) of images. Likewise, the steps 332 and/or 336 may be repeated based on the third subset of images or any subsequent set(s) of images.

The present disclosure encompasses embodiments of the method 300 that comprise more or fewer steps than those described above, and/or one or more steps that are different than the steps described above.

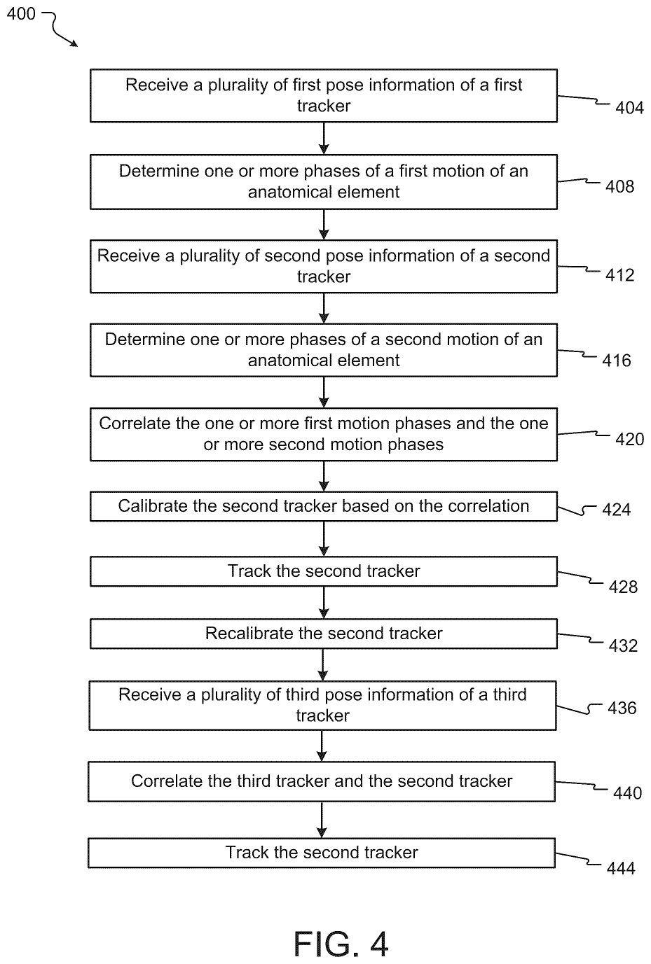

FIG. 4 depicts a method 400 that may be used, for example, for calibrating a tracking device such as the second tracking device 140, for example.

The method 400 (and/or one or more steps thereof) may be carried out or otherwise performed, for example, by at least one processor. The at least one processor may be the same as or similar to the processor(s) 104 of the computing device 102 described above. The at least one processor may be part of a robot (such as a robot 114) or part of a navigation system (such as a navigation system 118). A processor other than any processor described herein may also be used to execute the method 400. The at least one processor may perform the method 400 by executing elements stored in a memory such as the memory 106. The elements stored in memory and executed by the processor may cause the processor to execute one or more steps of a function as shown in method 400. One or more portions of a method 400 may be performed by the processor executing any of the contents of memory, such as image processing 120, tracking 122, registration 124, and/or reconstruction 128.

The method 400 comprises receiving a plurality of first pose information of a first tracker (step 404). The step 404 may be the same as or similar to the step 308 of the method 300 above.

The method 400 also comprises determining one or more phases of a first motion of an anatomical element (step 408). The step 408 may be the same as or similar to the step 312 of the method 300 above.

The method 400 comprises receiving a plurality of second pose information of a first tracker (step 412). The step 412 may be the same as or similar to the step 316 of the method 300 above.

The method 400 also comprises determining one or more phases of a second motion of the anatomical element (step 416). The step 416 may be the same as or similar to the step 320 of the method 300 above.

The method 400 also comprises correlating the one or more first motion phases and the one or more second motion phases (step 420). As previously described, the one or more first motion phases may correlate to one or more motion phases of a cardiac cycle and the one or more second motion phases may correlate to one or more motion phases of a respiratory cycle (or vice versa). The one or more first motion phases may be correlated to the one or more second motion phases based on, for example, the plurality of first pose information and the plurality of second pose information. For example, one first pose of the plurality of first pose information may be correlated with one second pose of the plurality of second pose information at a time stamp. By correlating the pose information, the phase(s) may also be correlated to each other. For example, a pose of the first tracker at an inspiration step of the respiratory cycle can be correlated to a pose of the second tracker at the inspiration step. Similarly, a pose of the first tracker at a diastole motion of the cardiac cycle can be correlated to a pose of the second tracker at the diastole motion.

It will be appreciated that in some embodiments, the patient may be instructed to inhale or exhale at certain intervals while the plurality of first pose information and the plurality of second pose information are being obtained. Thus, the first pose information and the second pose information can be more easily correlated with the patient's known inhalation and exhalation.

The method 400 also comprises calibrating the second tracker based on the correlation (step 424). The second tracker can be calibrated using the correlation such that the second tracker can be tracked to determine motion of the anatomical element without tracking the first tracker. In other words, the second tracker can be used to determine a phase or motion of the anatomical element.

The method 400 also comprises tracking the second tracker (step 428). The second tracker can be tracked by, for example, a navigation system such as the navigation system 118. As previously described, the second tracker can be tracked and used determine the phase(s) that the anatomical element is in based on the pose of the second tracker.

The method 400 also comprises recalibrating the second tracker (step 432). Recalibrating the tracker may comprise repeating one or more of the steps 404, 408, 412, 416, 420, and 424.

The method 400 comprises receiving a plurality of third pose information of a third tracker (step 436). The step 436 may be the same as or similar to the step 308 of the method 300 above. The third tracker may be the same as or similar to the third tracker 142. The third tracker may be positioned on the patient and spaced from the second tracker. More specifically, the second tracker may be positioned on the patient in an area of high respiratory motion and the third tracker may be positioned on the patient in an area of low respiratory motion.

The method 400 comprises correlating the third tracker and the second tracker (step 440). The third tracker and the second tracker can be correlated based on the plurality of second pose information (received in, for example, the step 412) and the plurality of third pose information (received in, for example, the step 436). In other words, the motion of the second tracker can be mapped or correlated with the motion of the third tracker. After such correlation, the second tracker can be monitored relative to the third tracker to determine where the patient is in the respiratory cycle.

The method 400 comprises tracking the second tracker (step 444). The step 444 may be the same as the step 428, except that the second tracker is tracked relative to the third tracker.

The present disclosure encompasses embodiments of the method 400 that comprise more or fewer steps than those described above, and/or one or more steps that are different than the steps described above.

As noted above, the present disclosure encompasses methods with fewer than all of the steps identified in FIGS. 3 and 4 (and the corresponding description of the methods 300 and 400), as well as methods that include additional steps beyond those identified in FIGS. 3 and 4 (and the corresponding description of the methods 300 and 400). The present disclosure also encompasses methods that comprise one or more steps from one method described herein, and one or more steps from another method described herein. Any correlation described herein may be or comprise a registration or any other correlation.

The foregoing is not intended to limit the disclosure to the form or forms disclosed herein. In the foregoing Detailed Description, for example, various features of the disclosure are grouped together in one or more aspects, embodiments, and/or configurations for the purpose of streamlining the disclosure. The features of the aspects, embodiments, and/or configurations of the disclosure may be combined in alternate aspects, embodiments, and/or configurations other than those discussed above. This method of disclosure is not to be interpreted as reflecting an intention that the claims require more features than are expressly recited in each claim. Rather, as the following claims reflect, inventive aspects lie in less than all features of a single foregoing disclosed aspect, embodiment, and/or configuration. Thus, the following claims are hereby incorporated into this Detailed Description, with each claim standing on its own as a separate preferred embodiment of the disclosure.

Moreover, though the foregoing has included description of one or more aspects, embodiments, and/or configurations and certain variations and modifications, other variations, combinations, and modifications are within the scope of the disclosure, e.g., as may be within the skill and knowledge of those in the art, after understanding the present disclosure. It is intended to obtain rights which include alternative aspects, embodiments, and/or configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A system for correlating one or more images with one or more phases of motion of an anatomical element comprising:
a processor; and
a memory storing data for processing by the processor, the data, when processed, causing the processor to:
receive a plurality of images over a period of time;
receive a plurality of first pose information of a first tracker corresponding to pose information of the first tracker over the period of time;
determine one or more first motion phases of a first motion of the anatomical element based on the plurality of first pose information;
receive a plurality of second pose information of a second tracker corresponding to pose information of the second tracker over the period of time;
determine one or more second motion phases of a second motion of the anatomical element based on the plurality of second pose information; and
determine a subset of images from the plurality of images corresponding to a target first motion phase of the one or more first motion phases of the first motion and a target second motion phase of the one or more second motion phases of the second motion.

2. The system of claim 1, wherein the subset of images is a first subset of images, and wherein the memory stores further data for processing by the processor that, when processed, causes the processor to determine a second subset of images from the plurality of images corresponding to another target first motion phase of the one or more first motion phases of the first motion and another target second motion phase of the one or more second motion phases of the second motion.

3. The system of claim 2, wherein the memory stores further data for processing by the processor that, when processed, causes the processor to reconstruct a three-dimensional model based on at least one of the first subset of images or the second subset of images.

4. The system of claim 3, wherein the three-dimensional model is reconstructed in near real-time.

5. The system of claim 2, wherein the memory stores further data for processing by the processor that, when processed, causes the processor to display at least one of the first subset of images or the second subset of images.

6. The system of claim 1, wherein the first motion corresponds to a cardiac cycle and the second motion corresponds to a respiratory cycle.

7. The system of claim 6, further comprising a catheter positioned in the anatomical element, wherein the first tracker is positioned on the catheter and tracks movement of the catheter, and wherein the second tracker is positioned on the patient and tracks respiratory movement of the patient.

8. The system of claim 1, wherein the memory stores further data for processing by the processor that, when processed, causes the processor to obtain and display a subsequent plurality of images over a subsequent period of time at the target first motion phase and the target second motion phase.

9. A system for calibrating a tracker with one or more phases of motion of an anatomical element comprising:
a processor; and
a memory storing data for processing by the processor, the data, when processed, causing the processor to:
receive a plurality of first pose information of a first tracker over a period of time, the first tracker disposed in an anatomical element;
determine one or more first motion phases of a first motion of the anatomical element based on the plurality of first pose information;
receive a plurality of second pose information of a second tracker over the period of time, the second tracker tracking respiratory movement of a patient;
determine one or more second motion phases of a second motion of the anatomical element based on the plurality of second pose information; and
correlate the one or more first motion phases and the one or more second motion phases; and
calibrate the second tracker based on the correlation of the one or more first motion phases and the one or more second motion phases.

10. The system of claim 9, wherein the memory stores further data for processing by the processor that, when processed, causes the processor to track the second tracker.

11. The system of claim 9, wherein the second tracker is external to the patient.

12. The system of claim 9, wherein the memory stores further data for processing by the processor that, when processed, causes the processor to:
receive a second plurality of first pose information of the first tracker over a second period of time;
determine a second one or more first motion phases of a first motion of the anatomical element based on the second plurality of first pose information;
receive a second plurality of second pose information of the second tracker over the second period of time;
determine a second one or more second motion phases of a second motion of the anatomical element based on the second plurality of second pose information;
correlate the second one or more first motion phases and the second one or more second motion phases; and
recalibrate the second tracker based on the correlation of the second one or more first motion phases and the second one or more second motion phases.

13. The system of claim 9, wherein the memory stores further data for processing by the processor that, when processed, causes the processor to:
receive a plurality of images over the period of time; and
determine a subset of images from the plurality of images corresponding to a target first motion phase of the one or more first motion phases of the first motion and a target second motion phase of the one or more second motion phases of the second motion.

14. The system of claim 13, wherein the memory stores further data for processing by the processor that, when processed, causes the processor to reconstruct a three-dimensional model based on at least one of the first subset of images or the second subset of images.

15. The system of claim 13, wherein the memory stores further data for processing by the processor that, when processed, causes the processor to obtain and display a subsequent plurality of images over a subsequent period of time at the target first motion phase and the target second motion phase.

16. The system of claim 9, wherein the first motion corresponds to a cardiac cycle.

17. The system of claim 9, wherein the plurality of second pose information is received as sensor data from at least one of an electromagnetic tracker or an impedance tracker.

18. The system of claim 9, wherein the plurality of second pose information is received from a navigation system configured to track the second tracker.

19. The system of claim 9, wherein the memory stores further data for processing by the processor that, when processed, causes the processor to determine another subset of images from the plurality of images corresponding to another target first motion phase of the one or more first motion phases of the first motion and another target second motion phase of the one or more second motion phases of the second motion.

20. The system of claim 9, wherein the memory stores further data for processing by the processor that, when processed, causes the processor to:
receive a plurality of third pose information of a third tracker over the period of time, the third tracker positioned on the patient and spaced from the second tracker; and
correlate the third tracker and the second tracker.

21. A system for correlating one or more motions of an anatomical element comprising:
a first tracker disposed on a catheter, the catheter positioned in the anatomical element;
as second tracker disposed on a patient;
a processor; and
a memory storing data for processing by the processor, the data, when processed, causing the processor to:
receive a plurality of first pose information of the first tracker over a period of time, the first pose information corresponding to pose information of the catheter;
determine one or more first motion phases of a first motion of the anatomical element based on the plurality of first pose information;
receive a plurality of second pose information of the second tracker over the period of time;
determine one or more second motion phases of a second motion of the anatomical element based on the plurality of second pose information; and
correlate the one or more first motion phases and the second motion phases.
